(19) United States
(12) Patent Application Publication
(10) Pub. No.: US 2006/0008833 A1
(43) Pub. Date: Jan. 12, 2006

(54) Method for long, error-reduced DNA synthesis

(76) Inventor: Joseph M. Jacobson, Newton, MA (US)

Correspondence Address: MORRISON ULMAN, WOODSIDE IP GROUP 1900 EMBARCADERO ROAD SUITE 209, PALO ALTO, CA, 94303-3327, US

(21) Appl. No.: 11/178,151

(22) Filed: Jul. 8, 2005

Related U.S. Application Data
(60) Provisional application No. 60/587,306, filed on Jul. 12, 2004.

(51) Int. Cl. C12Q 1/68; G06F 19/00; C12P 19/34

(52) U.S. Cl. 435/6; 435/91.2; 702/20

(57) ABSTRACT

A method for synthesizing a long, error-corrected DNA construct is disclosed. In the method, error-containing subregions of a long DNA sequence are replaced by repair oligonucleodides that are short enough that the probability of any one of them containing an error is less than one. Repeated repair cycles lead to a long DNA construct with very few remaining errors.

### RELATED APPLICATIONS

This application claims priority benefit of U.S. 60/587,306, filed on Jul. 12, 2004, and incorporated herein by reference.

### TECHNICAL FIELD

The invention relates generally to synthesis of long sequences of DNA.

### INTRODUCTION

Recently there has been considerable interest in the synthesis of sequences of DNA of gene length (˜1-2 kilobases) up to the size of small bacterial genomes (˜several megabases) concatenated from a series of synthetic oligonucleotides. Unfortunately the error rate of the best chemical syntheses for such synthetic oligonucleotides (acid labile or photo labile protection group chemistries) are typically on order of 1 error per 100 nucleotides making the resulting long constructs highly error laden.

One approach which has been employed by Venter et al. (Proceedings of the National Academy of Sciences, vol. 100, p. 15440-15445, Dec. 23, 2003, incorporated herein by reference) is to use best practices in synthesizing precursor oligonucleotides typically by co-synthesizing the complimentary oligonucelotides and running a thermally denaturing gel. Such practices can yield starting oligonucleotides with error rates of about 1 per 1000. As a next step small functional constructs such as viral genomes (˜5 Kb) can be constructed and tested for viability. In such a case a typical 5 Kb construct is likely to have 5 errors. However if on average there is a single error per 1000 bases then in any 500 base region there is a probability of ˜½ of having an error in that region. Thus for a 5 Kb construct consisting of ten 500-base regions there is a probability of (½)¹⁰= 1/1024 of creating the correct 5 Kb sequence. If one has a functional screen, such as the viability of the construct (e.g. viral infectivity) then one can pick out the correct construct from a colony. Alternatively one can randomly sequence members of the colony to be sequenced. (Note that one would have to sequence approximately 1024 members from a colony to find a 5 Kb sequence which was error free.) Unfortunately, although this approach is successful for shorter sequences, as the sequence length gets larger there is a high likelihood that no fully correct sequence exists in the pool of synthesized sequences. In order to synthesize such large sequences it is desirable to correct those errors which are found as opposed to merely sort them. One means of correcting sequence errors is to synthesize new oligonucleotides to replace regions which contain an error by means of site directed mutagenesis.

In co-pending application number U.S. Ser. No. 10/990,939 filed 11-17-2004 and claiming priority benefit of application number U.S. 60/520,751 filed 11-17-2003 both entitled “Nucleotide Sequencing via Repetitive Single Molecule Hybridization” and both incorporated herein by reference, we described the utility of using site directed mutagenesis to correct errors in a synthetic DNA construct found by sequencing. Subsequently, Venter et al. (Proceedings of the National Academy of Sciences, vol. 100, p. 15440-15445, Dec. 23, 2003, incorporated herein by reference) described the utility of using site directed mutagenesis to repair small numbers of remaining errors as a final clean up step in fabrication. Although useful, both of these approaches suffer from the fact that the repair oligos themselves have the same native error rate as the build oligos did initially.

Here we disclose a means for fabricating long DNA constructs assembled from imperfect oligos by means of repetitive cycling of the steps consisting of: [1] yes/no sequence verification in each subregion of the long DNA construct; [2] fabrication of repair oligos predicated on the outcome of such sequence verification; and, [3] replacement of error-containing subregions of the DNA construct with such repair oligos. A preferred means for yes/no sequence verification is by means of a hybridization array. A preferred means of replacement of error-containing regions with repair oligos is by site directed mutagenesis.

### SUMMARY

An aspect of the invention is a method for correcting errors in the synthesis of long sequences of DNA. In this approach an initial long DNA sequence is synthesized by means of creating an array of overlapping build oligonucleotides (e.g. 70 mers) using conventional array synthesis techniques. Next these oligos are released from the surface and allowed to hybridize to form a longer ‘walked up’ sequence. Using PCR assembly or ligase assembly the ‘walked up’ sequence can by covalently stitched together to form a longer sequence of double or single stranded DNA. Such a sequence will still possess (at best) the native synthetic error rate of the build oligo 1:100. This long DNA sequence is then incubated on a complimentary chip-based hybridization array to undergo yes/no sequence verification in each subregion (e.g. 35 nucleotide span) of the long DNA construct. Using this information a new repair oligo array is fabricated in which a repair oligo is synthesized for each subregion found to contain an error. Such repair oligos can then correct for such errors via the approach of site directed mutagenesis. If the appropriate sub region size is chosen (i.e. a size for which the probability of an error is less than one and preferably ˜½) repetition of this process yields a convergence toward an error free synthesized long DNA sequence.

Note that in certain cases one may wish to only synthesize a single molecule of any given oligo (and then amplify it if need be) so that there does not exist a population of errors within any one type of oligo.

### BRIEF DESCRIPTION OF THE DRAWINGS

The drawings are heuristic for clarity. The foregoing and other features, aspects and advantages of the invention will become better understood with regard to the following descriptions, appended claims and accompanying drawings in which:

FIG. 1 is a schematic drawing of an oligonucleotide chip with build oligos showing nucleotide level detail.

FIG. 2 is a schematic drawing of an oligonucleotide chip with build oligos.

FIG. 3A is a schematic drawing of build oligos which have been released from a chip and have hybridized (‘walked up’) to form a longer double stranded construct.

FIG. 3B is a schematic drawing of a double stranded long DNA construction from build oligos which have hybridized and then been ligated.

FIG. 4 is a schematic of a long single stranded DNA construct constructed from build oligos introduced onto a gene chip to analyze the presence or absence of particular base sequences in the single stranded DNA construct.

FIG. 5 is a schematic of an oligonucleotide chip with repair oligos.

FIG. 6 is a flowchart of steps for fabricating nearly perfect long DNA constructs from imperfect oligonucletides.

FIG. 7 is a table indicating the number of cycles, M*, of sequencing and repair required to build a nearly perfect long DNA construct.

### DETAILED DESCRIPTION

Described below is a preferred method for carrying the construction of a long, relatively error-free DNA construct from error-containing oligos.

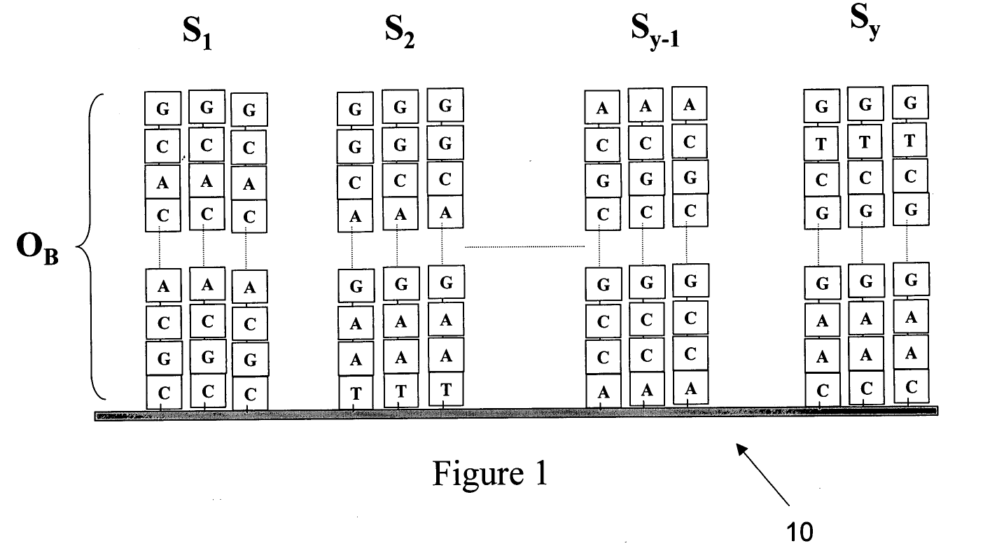

Referring to FIG. 1 a build oligonucleotide chip 10 with build oligo spots S1, S2 etc. of length O_{B} nucleotides (e.g. O_{B}=68; typically O_{B} will be set to twice the subregion size Q—see below) may be fabricated by standard means for fabricating DNA chips. Such oligos can be suitably designed that they can be released from the surface and further that they posses partially overlapping complimentary sequences such that when released they assemble into longer double stranded DNA sequences. We note that within any one build oligo spot (e.g. S1), the sequence of individual oligos can have variations due to errors in synthesis within a single spot.

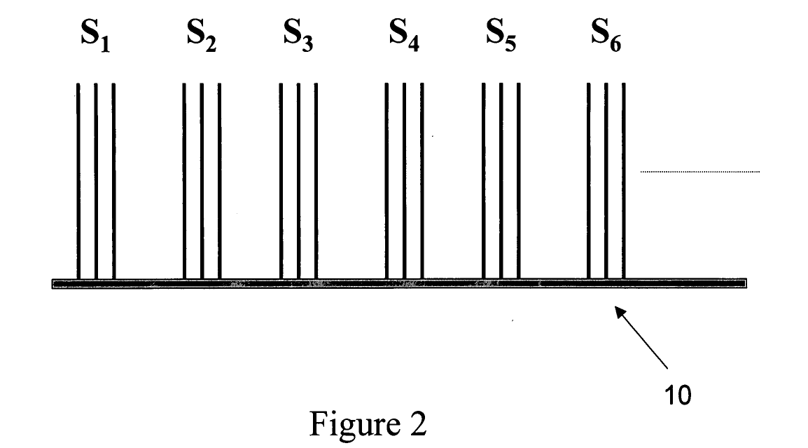

Referring to FIG. 2 as an example, a build oligonucleotide chip 10 is fabricated with build oligo spots S1, S2, S3, S4, S5, S6 designed to hybridize into a longer DNA construct when released from the chip.

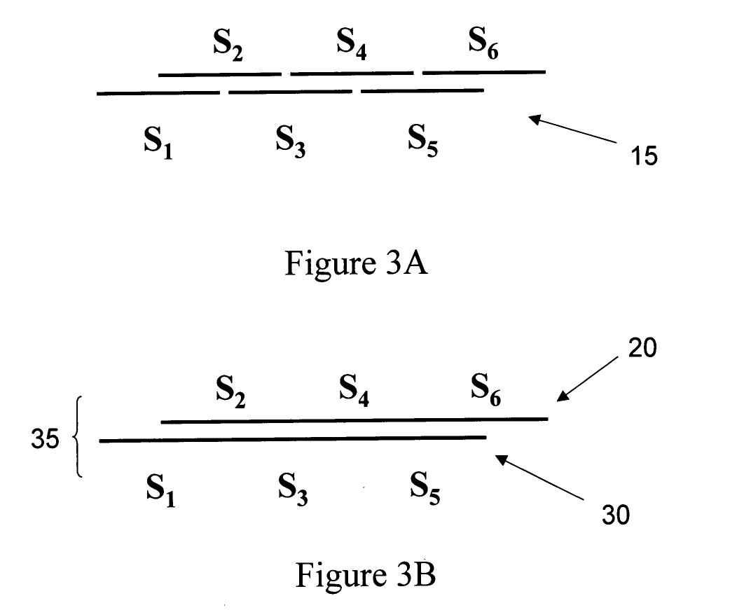

Oligos, S1-S6, may then be released from the chip and assembled into a longer double stranded DNA contruct (15 in FIG. 3A). The construct may further be ligated with ligase to form covalent top (20) and bottom (30) long DNA strands (FIG. 3B) together comprising a long DNA construct 35. It is important for future steps that if construct 35 need be amplified it is done by amplifying from a single initial copy (either by PCR or cloning) so that there do not exist distributions of errors within the long DNA construct.

At this point the DNA strands still possess the native error rate of the initial oligonucleotides. Consider the example where the native synthetic error rate for on-chip oligonucleotide synthesis, ε, is 0.98. In this case the probability of an error in any given subregion which is Q nucleotides in length is (1−ε)^{Q}. For convenience we can choose the length, Q, of our subregions such that there is a probability of ½ of there being an error in any given sub-region. In our example Q=34 bases. Typically O_{B} is set to be 2Q.

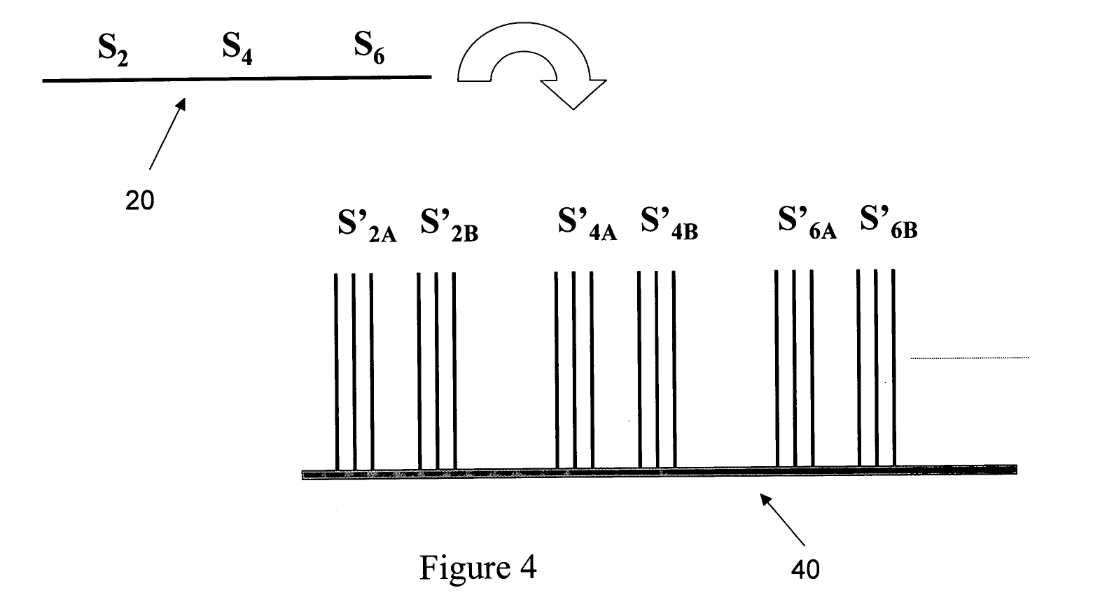

We now wish to query our long DNA construct to see whether in each subregion of Q bases we have an error as compared to the initially intended sequence. This can readily be carried out by means of dehybridizing our long double stranded DNA construct (FIG. 3B) into a single stranded DNA construct strand (e.g. top strand 20—FIG. 4) and then, referring to FIG. 4 exposing it to a hybridization chip array 40 containing complimentary oligos S′_{2A}, S′_{2B}, S′_{4A}, S′_{4B} and S′_{6A}, S′_{6B} in which S′_{2A} is complimentary to the first half of S₂ and S′_{2B} is complimentary to the second half of S₂ etc. Note that the length of the oligos on the hybridization array are typically Q in length and shorter than O_{B}. If there is an error in the DNA construct strand, for example in the first half S₄ then there will be less prevalent binding of the DNA construct strand to the corresponding S′_{4A} spot on the hybridization array chip. Such lack of binding can be read out by suitably fluorescently tagged DNA construct strands.

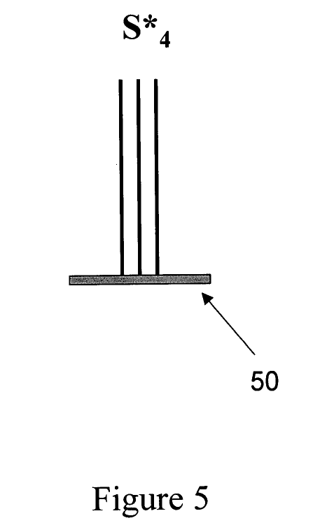

In order to repair errors that become known from binding to the hybridization array, such data may be used to direct the synthesis of repair oligos, typically of length Q (see FIG. 5). Such oligos may then be used to repair errors in the long DNA construct by means of site directed mutagenesis. It is important to note that for each repair oligo we do not wish to have sequence variation: thus we can either amplify up from a single repair oligo or clone it into an organism and amplify the oligo in-vivo.

An alternative approach to site directed mutagenesis is to shear or enzymatically cut the long DNA construct into smaller pieces and incubate them in a population of repair oligos (all repair oligos of each type being identical as noted above) and then to carry out reassembly by means of polymerase chain assembly in the presence of an abundance of repair oligo.

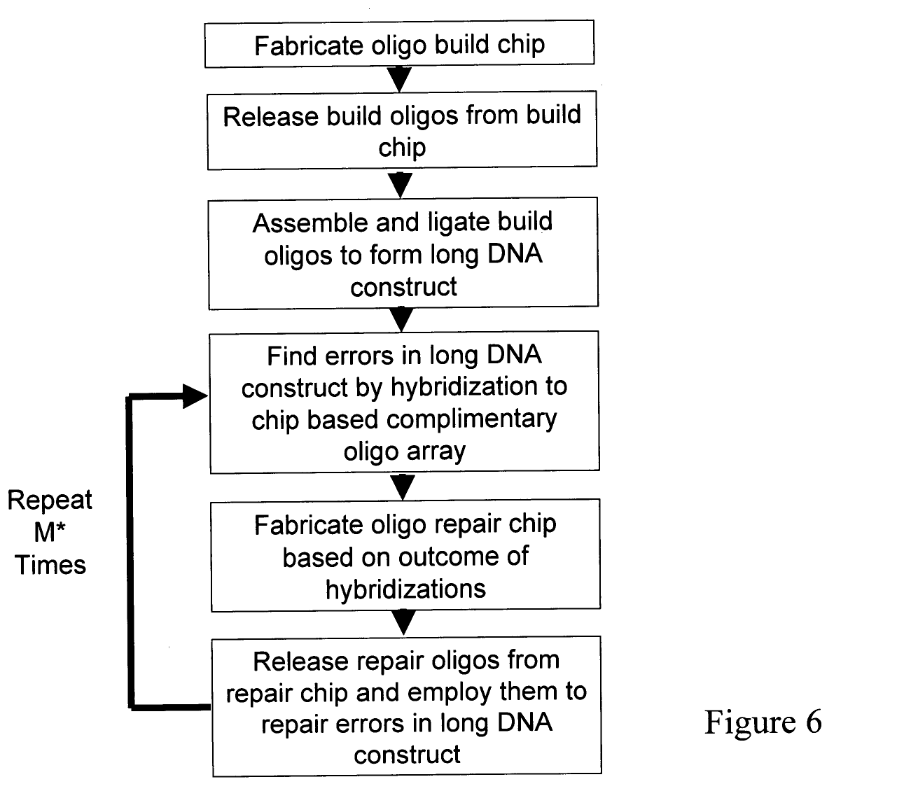

FIG. 6 shows a flowchart of the steps for fabricating nearly perfect long DNA constructs from imperfect oligonucletides as delineated above and further comprising repetition of the last 3 steps for M* cycles until convergenge to a nearly perfect construct is achieved.

The required number of cycles, M*, may be calculated as follows:
M*=−Log[N(1−ε)]/Log[1−Pₘ/2] where N is the length of the desired long DNA construct, ε is the per-base error rate for oligonucleotide synthesis, and Pₘ is the probability of the repair oligo properly replacing the native error-containing region via site directed mutagenesis.

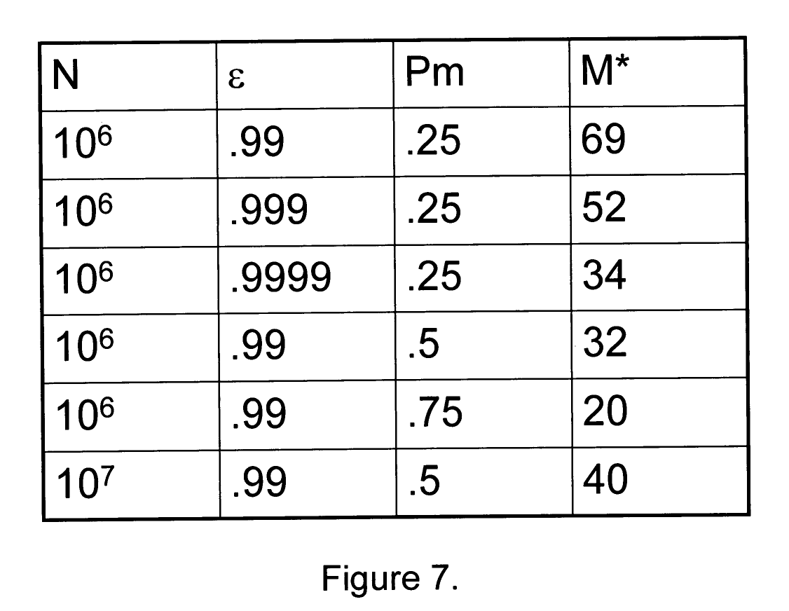

FIG. 7 is a table indicating the number of cycles, M*, of sequencing and repair required to build a nearly perfect long DNA construct of length N. As can be seen from the table both Pₘ and ε strongly affect the number of cycles M* which are required. Alternatives to site directed mutagenesis discussed above may have a strong beneficial effect on the effective Pₘ. Similarly, pre-purification of the build oligos by thermal gel shift or other enzymatic means can greatly increase the effective ε to as high as ε=0.9999.

While the invention has been described in connection with what are presently considered to be the most practical and preferred embodiments, it is to be understood that the invention is not limited to the disclosed embodiments and alternatives set forth above, but on the contrary is intended to cover various modifications and equivalent arrangements included within the scope of the following claims.

I claim:

1. A method for synthesizing error-corrected DNA constructs comprising the steps of:
[A] synthesizing a set of oligonucleotides, of which at least one oligonucleotide contains an error;
[B] assembling the oligonucleotides into a longer DNA construct which contains at least one error;
[C] testing for errors within subregions of the longer DNA construct;
[D] using information from testing to direct the synthesis of one or more repair oligonucleotides; and,
[E] using the repair oligonucleotides to repair errors in the longer DNA construct.

2. The method of claim 1 in which the testing step [C] is carried out by sequencing by hybridization.

3. The method of claim 1 in which the repair step [E] is carried out by site directed mutagenesis.

4. The method of claim 1 in which the repair step [E] is carried out by polymerase chain assembly in the presence of repair oligonucleotides.

5. The method of claim 1 in which the testing [C], using information [D] and repair [E] steps are repeated two or more times.

6. The method of claim 1 in which the oligonucleotides in the synthesizing step [A] are created on a chip.

7. The method of claim 1 in which the synthesis of repair oligonucleotides in step [D] consists of the synthesis of one or a few molecules of any one sequence of oligonucleotide.

8. The method of claim 1 in which the subregions tested in testing step [C] are shorter in length than the oligonucleotides in synthesized in step [A].

9. The method of claim 1 in which the subregions tested in testing step [C] are between 0.1 and 0.9 times the length of the oligonucleotides synthesized in step [A].

10. A method for synthesizing error-corrected DNA constructs comprising the steps of:
[A] synthesizing a set of oligonucleotides, at least one of which contains an error;
[B] assembling the oligonucleotides into a longer DNA construct which contains at least one error;
[C] testing for errors within subregions of the longer DNA construct;
[D] using information from testing to direct the synthesis of one or more repair oligonucleotides;
[E] using such repair oligonucleotides to repair errors in the longer DNA construct; and,
[F] repeating the testing [C], using information [D] and repair [E] steps until less than 1 error per 1000 oligonucleotides in the longer DNA construct remain.

11. A method for correcting a long DNA sequence comprising the steps of:
[A] synthesizing a long DNA sequence;
[B] replacing error-containing subregions of the DNA sequence with replacement subregions, wherein the lengths of the subregions are short enough that the probability of an error occurring in any particular replacement subregion is less than one; and,
[C] repeating step [B] until the long DNA sequence contains less than one error per thousand oligonucleotides.

12. The method of claim 12 wherein the lengths of the subregions are short enough that the probability of an error occurring in any particular replacement subregion is less than one-half.
